SEQUENCE LISTING (5 sequences)

SEQ ID NO: 1 - DNA, 2058 bp, Homo sapiens
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataa

SEQ ID NO: 2 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 3 - DNA, 3427 bp, Homo sapiens
ctcgcaggctaggaacccgaggccaagagctgcagccaaagtcacttgggtgcagtgtactccctcactagcccgctcgagaccctaggatttgctccaggacacgtacttagagcagccaccgcccagtcgccctcacctggattacctaccgaggcatcgagcagcggagtttttgagaaggcgacaagggagcagcgtcccgaggggaatcagcttttcaggaactcggctggcagacgggacttgcgggagagcgacatccctaacaagcagattcggagtcccggagtggagaggacaccccaagggatgacgcctgcgtcccggagcgcctgtcgctgggcgctactgctgctggcggtactgtggccgcagcagcgcgctgcgggctccggcatcttccagctgcggctgcaggagttcgtcaaccagcgcggtatgctggccaatgggcagtcctgcgaaccgggctgccggactttcttccgcatttgccttaagcacttccaggcaaccttctccgagggaccctgcacctttggcaatgtctccacgccggtattgggcaccaactccttcgtcgtcagggacaagaatagcggcagtggtcgcaaccctctgcagttgcccttcaatttcacctggccgggaaccttctcactcaacatccaagcttggcacacaccgggagacgacctgcggccagagacttcgccaggaaactctctcatcagccaaatcatcatccaaggctctcttgctgtgggtaagatttggcgaacagacgagcaaaatgacaccctcaccagactgagctactcttaccgggtcatctgcagtgacaactactatggagagagctgttctcgcctatgcaagaagcgcgatgaccacttcggacattatgagtgccagccagatggcagcctgtcctgcctgccgggctggactgggaagtactgtgaccagcctatatgtctttctggctgtcatgagcagaatggttactgcagcaagccagatgagtgcatctgccgtccaggttggcagggtcgcctgtgcaatgaatgtatcccccacaatggctgtcgtcatggcacctgcagcatcccctggcagtgtgcctgcgatgagggatggggaggtctgttttgtgaccaagatctcaactactgtactcaccactctccgtgcaagaatggatcaacgtgttccaacagtgggccaaagggttatacctgcacctgtctcccaggctacactggtgagcactgtgagctgggactcagcaagtgtgccagcaacccctgtcgaaatggtggcagctgtaaggaccaggagaatagctaccactgcctgtgtcccccaggctactatggccagcactgtgagcatagtaccttgacctgtgcggactcaccctgcttcaatgggggctcttgccgggagcgcaaccaggggtccagttatgcctgcgaatgcccccccaactttaccggctctaactgtgagaagaaagtagacaggtgtaccagcaacccgtgtgccaatggaggccagtgcctgaacagaggtccaagccgaacctgccgctgccggcctggattcacaggcacccactgtgaactgcacatcagcgattgtgcccgaagtccctgtgcccacgggggcacttgccacgatctggagaatgggcctgtgtgcacctgccccgctggcttctctggcaggcgctgcgaggtgcggataacccacgatgcctgtgcctccggaccctgcttcaatggggccacctgctacactggcctctccccaaacaacttcgtctgcaactgtccttatggctttgtgggcagccgctgcgagtttcccgtgggcttgccacccagcttcccctgggtagctgtctcgctgggcgtggggctagtggtactgctggtgctgctggtcatggtggtagtggctgtgcggcagctgcggcttcggaggcccgatgacgagagcagggaagccatgaacaatctgtcagacttccagaaggacaacctaatccctgccgcccagctcaaaaacacaaaccagaagaaggagctggaagtggactgtggtctggacaagtccaattgtggcaaactgcagaaccacacattggactacaatctagccccgggactcctaggacggggcagcatgcctgggaagtatcctcacagtgacaagagcttaggagagaaggtgccacttcggttacacagtgagaagccagagtgtcgaatatcagccatttgctctcccagggactctatgtaccaatcagtgtgtttgatatcagaagagaggaacgagtgtgtgattgccacagaggtataaggcaggagcctactcagacacccagctccggcccagcagctgggccttccttctgcattgtttacattgcatcctgtatgggacatctttagtatgcacagtgctgctctgcggaggaggagggaatggcatgaactgaacagactgtgaacccgccaagagttgcaccggctctgcacacctccaggagtctgcctggcttcagatgggcagccccgccaagggaacagagttgaggagttagaggagcatcagttgagctgatatctaaggtgcctctcgaacttggacttgctctgccaacagtggtcatcatggagctcttgactgttctccagagagtggcagtggccctagtgggtcttggcgctgctgtagctcctgtgggcatctgtatttccaaagtgcctttgcccagactccatcctcacagctgggcccaaatgagaaagcagagaggaggcttgcaaaggataggcctcccgcaggcagaacagccttggagtttggcattaagcaggagctactctgcaggtgaggaaagcccgaggaggggacacgtgtgactcctgcctccaaccccagcaggtggggtgccacctgcagcctctaggcaagagttggtccttcccctggtcctggtgcctctgggctcatgtgaacagatgggcttagggcacgccccttttgccagccaggggtacaggcctcactggggagctcagggccttcatgctaaactcccaataagggagatggggggaagggggctgtggcctaggcccttccctccctcacacccatttttgggcccttgagcctgggctccaccagtgcccactgttgccccgagaccaaccttgaagccgattttcaaaaatcaataatatgaggttttgttttgtagtttattttggaatctagtattttgataatttaagaatcagaagcactggcctttctacattttataacattattttgtatataatgtgtatttataatatgaaacagatgtgtacataaaaaaaaaaaaaaaaaaaaaaaaaaaaaa

SEQ ID NO: 4 - protein, 686 aa, Homo sapiens
MTPASRSACRWALLLLAVLWPQQRAAGSGIFQLRLQEFVNQRGMLANGQSCEPGCRTFFRICLKHFQATFSEGPCTFGNVSTPVLGTNSFVVRDKNSGSGRNPLQLPFNFTWPGTFSLNIQAWHTPGDDLRPETSPGNSLISQIIIQGSLAVGKIWRTDEQNDTLTRLSYSYRVICSDNYYGESCSRLCKKRDDHFGHYECQPDGSLSCLPGWTGKYCDQPICLSGCHEQNGYCSKPDECICRPGWQGRLCNECIPHNGCRHGTCSIPWQCACDEGWGGLFCDQDLNYCTHHSPCKNGSTCSNSGPKGYTCTCLPGYTGEHCELGLSKCASNPCRNGGSCKDQENSYHCLCPPGYYGQHCEHSTLTCADSPCFNGGSCRERNQGSSYACECPPNFTGSNCEKKVDRCTSNPCANGGQCLNRGPSRTCRCRPGFTGTHCELHISDCARSPCAHGGTCHDLENGPVCTCPAGFSGRRCEVRITHDACASGPCFNGATCYTGLSPNNFVCNCPYGFVGSRCEFPVGLPPSFPWVAVSLGVGLVVLLVLLVMVVVAVRQLRLRRPDDESREAMNNLSDFQKDNLIPAAQLKNTNQKKELEVDCGLDKSNCGKLQNHTLDYNLAPGLLGRGSMPGKYPHSDKSLGEKVPLRLHSEKPECRISAICSPRDSMYQSVCLISEERNECVIATEV

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2008/0181893 A1
(43) Pub. Date: Jul. 31, 2008

(54) Therapeutic methods for treating vascular eye disorders with DII4 antagonists

(76) Inventors: Ivan B. Lobov, New York, NY (US); Nicholas Papadopoulos, LaGrangeville, NY (US); Stanley J. Wiegand, Croton-on-Hudson, NY (US)

Correspondence Address: REGENERON PHARMACEUTICALS, INC, 777 OLD SAW MILL RIVER ROAD, TARRYTOWN, NY, 10591, US

(21) Appl. No.: 11/890,741

(22) Filed: Aug. 7, 2007

Related U.S. Application Data
(60) Provisional application No. 60/836,003, filed on Aug. 7, 2006.

(51) Int. Cl. A61K 39/395; A61P 27/02

(52) U.S. Cl. 424/135.1; 424/133.1; 424/141.1; 424/130.1

(57) ABSTRACT

A therapeutic method for treating ischemic or vascular disorders by administering an agent capable of inhibiting human delta-like ligand 4 (Dll4) activity to a subject in need thereof. In one embodiment, the agent is an anti-Dll4 antibody or antibody fragment capable of inhibiting the binding of Dll4 to a Notch receptor. The method of the invention is useful for treating eye disorders such as ischemic retinopathy, diabetic retinopathy, age related macular degeneration, corneal neovascularization, neovascular glaucoma, or retinopathy of prematurity. The method is also useful or treating ischemic or vascular disorders such as ischemic injury, cerebral ischemia, cardiac ischemia, ischemic conditions affecting the limbs and other organs or tissues, arteriovenous malformations, wound healing, organ or tissue transplantation, placental insufficiency, arterial narrowing and occlusion, atherosclerosis, and systemic or pulmonary hypertension.

### CROSS-REFERENCE TO RELATED APPLICATIONS

This application claims the benefit under 35 USC §119(e) of U.S. Provisional application 60/623,658 filed 29 Oct. 2004, which application is herein specifically incorporated by reference in its entirety.

### FIELD OF INVENTION

This invention relates generally to methods for treating vascular and ischemic disorders by administering compositions that inhibit Dll4 interaction with Notch receptors, and/or Notch receptor activation. More specifically, this invention relates to methods of treating eye vascular and/or ischemic disorders by administering compositions that inhibit Dll4 interaction with Notch receptors.

### BACKGROUND OF THE INVENTION

Angiogenesis is a fundamental process required for the normal development and growth of tissues and organs, and involves formation of new blood vessels from pre-existing vessels. Angiogenesis and blood vessel homeostasis are tightly controlled through a highly regulated system of angiogenic modulators. Deviation from such a tight control often leads to or is associated with disease.

While unregulated “excessive” or aberrant angiogenesis is characteristic of numerous disease states, insufficient angiogenesis, loss of blood vessels or functional or structural blood vessel occlusion can also be a serious medical problem. Promoting angiogenesis and/or preventing regression of existing blood vessels is desirable in situations where tissue becomes ischemic, for example in retinal, cerebral, cardiovascular and limb ischemia and in conditions where a vascular supply must be established, re-established, enhanced or expanded, for example in wound healing and after tissue or organ transplantation, or to stimulate establishment of collateral vessels or otherwise increase the perfusion of a tissue or organ with inadequate circulation.

The Notch-signaling pathway is a system for cell-to-cell communication used by a wide range of eukaryotes for many biological processes, such as differentiation, proliferation, and homeostasis (Artavanis-Tsakonas et al. (1999) Science 284:770-776). Notch signaling has also been implicated in the control of vascular development (Iso et al. (2003) Arterioscler Thromb Vasc Biol. 23:543-553).

Delta-like 4 (Dl4) or delta-like ligand 4 (Dll4) (hereinafter “Dll4”) is a member of the Delta family of Notch ligands which exhibits highly selective expression by vascular endothelium (Shutter et al. (2000) Genes Dev. 14:1313-1318). Dll4 is a ligand for Notch receptors, including Notch1 and Notch 4. The nucleic acid and amino acid sequences for human and mouse Dll4 are shown in SEQ ID NO:1-2 and SEQ ID NO:3-4, respectively. Gene-targeted Dll4 mice have been generated (see, for example, Duarte et al. (2004) Genes Dev. 18:2474-2478; Krebs et al. (2004) Genes Dev. 18:2469-2473: Gale et al. (2004) Proc Natl Acad Sci USA 101:15949-15954). These studies showed that Dll4 was highly expressed on developing blood vessels in the mouse embryo, and that genetic deletion of even a single Dll4 allele resulted in embryonic lethality, associated with marked vascular abnormalities.

### BRIEF SUMMARY OF THE INVENTION

The present invention relates to methods of treating diseases affecting the cardiovascular system with agents capable of blocking the interaction of Dll4 with its receptors (“Dll4 antagonists”). The experimental results provided herein support the use of Dll4 antagonists for treating diseases characterized by pathological neovascularization and vascular insufficiency, where vascular insufficiency is due to the loss of blood vessels or functional vascular changes resulting in insufficient tissue perfusion. Dll4 antagonists are particularly useful for treating vascular disorders of the eye, for example, diabetic retinopathy, retinopathy of prematurity and other ocular diseases characterized by pathological angiogenesis and/or retinal ischemia, such as retinal vein or artery occlusion.

Pharmacological inhibition of Dll4 is shown to exert several beneficial effects in a model of ischemic retinopathy, including attenuation of blood vessel loss when applied at the time of an injury leading to blood vessel loss, and suppression of the development of pathological changes in the vasculature while enhancing the regrowth of more normal, functional blood vessels when applied following the establishment of ischemia.

Pharmacological agents which inhibit Dll4 signaling are expected to be similarly beneficial in treating other forms of ischemic injury, including cerebral ischemia, cardiac ischemia, and ischemic conditions affect the limbs and other body organs or tissues. Dll4 inhibitors are also expected to be clinically beneficial in other conditions requiring the establishment or re-establishment of a vascular supply or that would otherwise benefit from enhanced tissue perfusion. Examples of such conditions are arteriovenous malformations, wound healing, organ or tissue transplantation, and placental insufficiency. As shown in the experimental work reported below, Dll4 inhibition prevents arterial narrowing and occlusion, indicating that Dll4 inhibitors would also be effective in treating systemic or pulmonary hypertension, and related disorders.

Dll4 antagonists are now shown to be effective in promoting productive angiogenesis in both normal and pathological conditions. Specifically, blocking Dll4-Notch signaling is shown to enhance angiogenic sprouting and vascular endothelial cell proliferation in the developing retinal vasculature, and to suppress formation of pathological, ectopic neovascularization and the development of arteriovenous shunts in favor of more appropriate vessel formation in the ischemic retina. Moreover, the application of Dll4 blockers at the time of vessel injury is shown to markedly reduce the subsequent loss of blood vessels. These findings support the use of a Dll4 antagonist in the treatment of eye diseases characterized by vascular abnormalities, especially when accompanied by ischemia and loss of normal vessels. Examples of such eye diseases include ischemic retinopathies, such as age-related macular degeneration, central retinal vein occlusion or branch retinal vein occlusion, diabetic retinopathy, and retinopathy of prematurity.

In a first aspect, the invention features a method of treating an eye disorder or disease characterized by vascular abnormalities, comprising administering a Dll4 antagonist to a subject in need thereof. The method of the invention promotes growth of functional normal vessels, inhibits the growth of abnormal or disordered vessels and prevents pathological regression of blood vessels.

In one embodiment, the Dll4 antagonist is an antibody or antibody fragment which specifically binds Dll4 and blocks Dll4 binding to a Notch receptor (for example, Notch1 and Notch 4 receptors). In another embodiment, the Dll4 antagonist of the invention is a protein or protein fragment comprising the extracellular domain of Dll4 or a fragment thereof, which in specific embodiments may be fused to a multimerizing component and which binds Notch receptors without activating them, thereby blocking the actions of endogenous Dll4.

The eye disorder or disease treated by the method of the invention is a disorder of the ocular vasculature characterized by the presence of abnormal vessels and/or loss of normal vessels or normal vessel function. In specific embodiments, the condition or disorder treated includes as retinopathy of prematurity, ischemic retinopathy, retinal vein or artery occlusion, diabetic retinopathy, choroidal neovascularization, age related macular degeneration, corneal neovascularization, neovascular glaucoma or corneal transplantation.

The antibody or antibody fragment used in the method of the invention may be polyclonal or monoclonal, and may be humanized, chimeric, or fully human. Preferably the antibody is a fully human monoclonal antibody or monoclonal antibody fragment. The antibody fragment may be a single chain antibody, ScFv, an Fab, or an F(ab′)₂.

When the Dll4 antagonist is a protein or protein fragment, the fragment is preferably the extracellular domain of Dll4 or a fragment or modified fragment thereof, and may be fused to a multimerizing component. The multimerizing component is preferably an immunoglobulin domain, such as for example an Fc domain, e.g., a human Fc (SEQ ID NO:5). The protein may optionally comprise a signal sequence, which may be native to the cell, recombinant, or synthetic.

In a broader aspect, the method of the invention is useful to treat any ischemic disease or condition caused by insufficient blood supply due to blood vessel loss and/or poor perfusion, for example ischemic injury, cerebral ischemia, cardiac ischemia, ischemic conditions affecting the limbs and other organs or tissues, arteriovenous malformations, wound healing, organ or tissue transplantation, placental insufficiency, arterial narrowing and occlusion, atherosclerosis, and systemic or pulmonary hypertension.

Other objects and advantages will become apparent from a review of the ensuing detailed description.

### BRIEF SUMMARY OF THE FIGURES

FIG. 1. Genetic deletion of a single Dll4 allele increased numbers of angiogenic sprouts in the developing retinal vasculature. Numbers of sprouts were quantified in 100× microscopy fields. Results were significantly different at the p<0.00001 level.

FIG. 2. Intraocular delivery of Dll4-Fc increased numbers of proliferating BrdU-positive cells in the developing retinal vasculature. 4.15 mcg of mDll4-hFc or 5 mcg of human hFc control protein was injected intravitreally in 7 days old mouse pups. Retinal vasculature was analyzed 24 hours later. Numbers of BrdU positive cells were quantified in 200× microscopy fields. Results were significantly different at the p<0.05 level.

FIG. 3A-B. Intraocular delivery of Dll4-Fc or anti-Dll4 antibody promotes the regrowth of retinal vessels in mice with oxygen-induced ischemic retinopathy (OIR). (A) 0.48 mcg of mDll4-hFc or 0.5 mcg of human hFc control protein was injected intravitreally in 13 days old (postnatal day 13, or P13) OIR pups. Retinal vasculature was analyzed at P17. (B) 2.55 mcg of rabbit polyclonal anti-mDll4 antibody or 5 mcg of human hFc control protein was injected intravitreally at P13. Retinal vasculature was analyzed at P17. Avascular areas were measured in retinal flat-mounts. Results were significantly different at the p<0.0001 (A) and p<0.05 (B) levels.

FIG. 4A-B. Intraocular delivery of Dll4-Fc or anti-Dll4 antibody ameliorates formation of pathological neovascularization in OIR. (A) 0.48 mcg of mDll4-hFc or 0.5 mcg of human hFc control protein was injected intravitreally at P13. Retinal vasculature was analyzed at P17. (B) 2.55 mcg of rabbit polyclonal anti-mDll4 antibody or 5 mcg of human hFc control protein was injected intravitreally at P13. Retinal vasculature was analyzed at P17. Abnormal vascular areas (areas containing ectopic vascular “tufts”) were measured in retinal flat-mounts. Results were significantly different at the p<0.0001 (A) and p<0.05 (B) levels.

FIG. 5. Intraocular delivery of Dll4-Fc improves retinal perfusion. 0.48 mcg of mDll4-hFc or 0.5 mcg of human hFc control protein was injected intravitreally at P13. At P17 animals were perfused with tomato lectin labeled with Texas Red and retinal vasculature was analyzed at P17. Non-perfused vascular areas were measured in retinal flat-mounts. Results were significant at the p<0.0005 level.

FIG. 6A-B. Intraocular delivery of Dll4-Fc or anti-Dll4 antibody reduces retinal hypoxia/ischemia. (A) 4.1 mcg of hDll4-hFc or 5 mcg of human hFc control protein was injected intravitreally at P12. Hypoxyprobe™-1 was injected intraperitoneally one hour before sacrificing the animals. Retinal vasculature was analyzed at P17. Hypoxyprobe-labeled areas were measured in retinal flat-mounts. (B) 2.55 mcg of rabbit polyclonal anti-mDll4 antibody or 5 mcg of human hFc control protein was injected intravitreally at P13. Hypoxyprobe™-1 was injected intraperitoneally one hour before sacrificing the animals. Retinal vasculature was analyzed at P17. Hypoxyprobe-labeled areas were measured in retinal flat-mounts. Results were significantly different at the p<0.001 (A) and p<0.05 (B) levels.

FIG. 7. Genetic deletion of a single Dll4 allele reduces blood vessel loss induced by exposure to hyperoxia. Dll4^{+/lacZ} and littermate WT control mice were placed into the 75% oxygen chamber at P7. Retinal vasculature was analyzed in flat-mounts at P12. Results were significantly different at the p<0.05 levels.

FIG. 8A-B. Intraocular delivery of Dll4-Fc or anti-Dll4 antibody reduces or prevents blood vessel loss induced by exposure to hyperoxia. (A) 4.1 mcg of hDll4-hFc or 5 mcg of human hFc control protein was injected intravitreally at P8. Pups were placed into a 75% oxygen environment at P9. Retinas vasculature was analyzed at P10. (B) 2.55 mcg of rabbit polyclonal anti-mDll4 antibody or 5 mcg of human hFc control protein was injected intravitreally at P8. Pups were placed into a 75% oxygen environment at P9. Retinal vasculature was analyzed at P10. Results were significantly different at the p<0.00001 (A) and p<0.0001 (B) levels.

FIG. 9A-B. Intraocular delivery of Dll4-Fc or anti-Dll4 antibody reduces retinal vessel occlusion. (A) 4.1 mcg of hDll4-hFc or 5 mcg of human hFc control protein was injected intravitreally at P8. Pups were placed into a 75% oxygen environment at P9. Pups were perfused with fluorescent lectin and retinal vasculature was analyzed at P10. (B) 2.55 mcg of rabbit polyclonal anti-mDll4 antibody or 5 mcg of human hFc control protein was injected intravitreally at P8. Pups were placed into a 75% oxygen environment at P9. Pups were perfused with fluorescent lectin and retinal vasculature was analyzed at P10. Results were significantly different at the p<0.00001 (A) and p<0.0001 (B) levels.

FIG. 10. Systemic delivery of Dll4-Fc reduces or prevents retinal blood vessel loss. 4.1 mcg of hDll4-hFc or 5 mcg of human hFc control protein was injected intravitreally (ITV) or hDll4-hFc was injected intraperitoneally at a dose of 25 mg per kg of the body weight at P7. Pups were placed into a 75% oxygen environment at P8. Retinal vasculature was analyzed at P9. Results were significantly different at the p<0.0001 level.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated by reference in their entirety.

### Definitions

By the phrase “Dll4 antagonist” is meant an agent capable of inhibiting Dll4 biological activity by blocking the interaction of Dll4 with its receptors. Inhibition of Dll4 activity may be through inhibition of receptor activation either with an antibody to Dll4 or with a Dll4 analog or fragment which binds but does not activate the receptor (nonproductive binding). Common inhibitors include but are not limited to antibodies, soluble receptors, oligonucelotide aptamers, peptides or other small molecule antagonists and their derivatives, and modified Dll4 ligands which bind their Notch receptor but are unable to activate signaling through such binding. Other approaches include interfering with the expression of the gene encoding Dll4 or blocking Notch receptor activation, for example with siRNAs or gamma secretase inhibitors, respectively.

A “neutralizing” or “blocking” antibody, is intended to refer to an antibody whose binding to Dll4 results in inhibition of the biological activity of Dll4. This inhibition of the biological activity of Dll4 can be assessed by measuring one or more indicators of Dll4 biological activity. These indicators of Dll4 biological activity can be assessed by one or more of several standard in vitro or in vivo assays known in the art (see examples below). Preferably, the ability of an antibody to neutralize Dll4 activity is assessed by inhibition of Dll4 binding to a Notch receptor, such as Notch1 and Notch4.

### General Description

Notch signaling pathways are evolutionarily conserved, and play key roles in cell-fate determination and differentiation in many tissues during embryonic and postnatal development. Major components of the Notch pathway are expressed in the vasculature and genetic deletion of certain Notch pathway components, including Notch1, Notch1/Notch4, Jagged1, Dll1, Dll4, Hey1/Hey2 or presenilins, results in embryonic lethality associated with vascular remodeling defects. Although most of these genes are expressed in multiple tissue and cell types, Dll4 is largely restricted to the vascular endothelium, suggesting that Dll4 is a key ligand for Notch receptors in the vasculature.

During early embryonic development, genetic deletion of even a single Dll4 allele produces severe vascular abnormalities that result in embryonic lethality,in most mouse strains. Indeed, among the many genes involved in vasculogenesis and angiogenesis, haploid insufficiency has been reported to result in major vascular defects and embryonic lethality only for Dll4 and VEGF-A. Early embryonic lethality precludes most experimental manipulations, making it difficult to precisely understand the role of Dll4 during vascular development and in pathological settings.

To overcome this limitation, the effects of Dll4 gene deletion were studied in mice of the ICR strain, in which haplo-insufficiency produces only limited embryonic lethality. The vascular phenotype observed in these mutant mice was compared to that obtained in wild-type mice in which Dll4/Notch signaling was selectively inhibited by intravitreal injection of a soluble inhibitor of Dll4, Dll4-Fc, or a neutralizing polyclonal antibody against the extracellular domain of Dll4. For these experiments, the retina was selected as a model system in which to study Dll4 biology, because the retinal vasculature develops postnatally in a stereotypic manner that is highly organized, temporally and spatially.

The murine model of oxygen-induced ischemic retinopathy (OIR) is a well characterized model of pathological neovascularization associated with elevated expression of the essential pro-angiogenic factor, VEGF (Smith et al. 1994 Invest Ophthalmol Vis Sci 35:101-111; Neely et al. 1998 Am J. Pathol 153:665-670; Saint-Geniez et al. 2004 Int J Dev Biol 48:1045-1058) and thus relevant to pathological angiogenesis associated with diverse disease conditions (Ferrarra et al. 2005 Nature 438:967-974). Finally, the retinal vasculature is readily accessible to experimental manipulations, including intravitreal microinjections of experimental agents.

The experiments described below show that that during normal retinal vascular development, and in the OIR model, pharmacological suppression of Dll4/Notch signaling markedly enhances angiogenic sprouting and promotes the formation of a denser primary capillary network. Consistent with this, it was found that endogenous Dll4 expression is particularly prominent in the most active regions of vascular growth during both normal development and in the OIR model. Moreover Dll4 inhibition markedly suppressed formation of the abnormal vasculature and prevented blood vessel occlusion and regression.

### Dll4 Antagonists

Dll4 antagonists include antibodies to Dll4 and fragments thereof capable of blocking the binding of Dll4 to a Notch receptor, for example Notch1; and proteins or protein fragments comprising the extracellular domain of Dll4 which may be fused to a multimerizing component; peptides and peptibodies (see for example, US patent publication 2003/0229023 Oliner et al. herein specifically incorporated by reference in its entirety).

Dll4 antibodies. The phrase “immunoglobulin or antibody” as used herein refers to a mammalian, including human, polypeptide or protein comprising a framework region from an immunoglobulin gene or fragments thereof that specifically binds and recognizes an antigen, which, in the case of the present invention, is a Dll4 protein or portion thereof. If the intended antibody or antibody-like protein will be used as a mammalian therapeutic, immunoglobulin binding regions should be derived from the corresponding mammalian immunoglobulins. If the molecule is intended for non-therapeutic use, such as for diagnostics and ELISAs, the immunoglobulin binding regions may be derived from either human or non-human mammals, such as mice. The human immunoglobulin genes or gene fragments include the kappa, lambda, alpha, gamma, delta, epsilon, and mu constant regions, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Within each IgG class, there are different isotypes (eg. IgG₁, IgG₂, etc.) as well as allotypes thereof.

An exemplary immunoglobulin (antibody) structural unit of human IgG, comprises a tetramer. Each tetramer is composed of two identical pairs of polypeptide chains, each pair having one light chain (about 25 kD) and one heavy chain (about 50-70 kD). The N-terminus of each chain defines a variable region of about 100-110 or more amino acids primarily responsible for antigen recognition. The terms “variable light chain” (V_{L}) and variable heavy chain (V_{H}) refer to these light and heavy chains respectively.

Antibodies exist as intact immunoglobulins, or as a number of well-characterized fragments produced by digestion with various peptidases. For example, pepsin digests an antibody below the disulfide linkages in the hinge region to produce F(ab)′₂, a dimer of Fab which itself is a light chain joined to V_{H}—C_{H} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are described in terms of the digestion of an intact antibody, one of skill will appreciate that such fragments may be synthesized de novo either chemically or by using recombinant DNA methodology. Thus, the terms antibody, as used herein, also includes antibody fragments either produced by the modification of whole antibodies, or those synthesized de novo using recombinant DNA methodologies, or generated via display libraries such as phage, E. coli or yeast display libraries (see, for example, McCafferty et al. (1990) Nature 348:552-554).

Dll4 analogs or Protein fragments. The Dll4 antagonist may be a Dll4 fragment optionally connected to a multimerizing component. In specific embodiments, a Dll4 fragment may be fused to a multimerizing component such as an immunoglobulin domain, a truncated immunoglobulin domain, or an amino acid sequence between 1 to about 500 amino acids in length, optionally comprising at least one cysteine residue. In a preferred embodiment, the multimerizing component is an immunoglobulin domain, preferably an Fc domain, e.g., a human Fc (SEQ ID NO:5). The protein or protein fragment may optionally comprise a signal sequence, which may comprise any sequence known to a skilled artisan for directing secretion of a polypeptide or protein from a cell, including natural or synthetic sequences. Generally, a signal sequence is placed at the beginning or amino-terminus of the fusion protein of the invention. Such a signal sequence may be native to the cell, recombinant, or synthetic.

The extracellular domain of Dll4 is composed of a Delta/Serrate/Lag-2 (DSL) domain and eight epidermal growth factor (EGF)-like tandem repeats. Generally, the EGF domains are recognized as occurring at about position 218-251 (domain 1), 252-282 (domain 2), 284-322 (domain 3), 324-360 (domain 4), and 362-400 (domain 5), with the DSL domain at about position 173-217 and the N-terminal domain at about position 27-172 of hDll4 (SEQ ID NO:2). In specific embodiments, the Dll4 antagonist comprises about amino acid 27-172, 27-217, 218-400, 218-360, 218-322, or 218-282 of SEQ ID NO:2, optionally fused to hFc (SEQ ID NO:5).

### Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as a cow, pig, horse, chicken, cat, dog, etc., and is preferably a mammal, and most preferably human. In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant.

The methods of the invention may be advantageously performed by administration to the area in need of treatment by local administration, including intravitreal, intraocular, periocular, subconjunctival, juxtascleral, subtenon, or topical administration.

### Combination Therapies

In various embodiments, the method of the invention is accomplished with a Dll4 antagonist, such as a Dll4 antibody, in combination with one or more additional compounds or therapies or medical procedures. For example, suitable therapeutic agents for use in combination, either alternating or simultaneously, include fusion proteins capable of binding and inhibiting the activity of vascular endothelial growth factor (VEGF) (see U.S. Pat. Nos. 7,070,959 and 7,087,411, herein specifically incorporated by reference), immunosuppressive agents such as corticosteroids, dexamethasone, cyclosporin A, FK506, or anti-metabolic agents, (see Barker, N H, et al., (2000) Clin Exp Opthal 28:357-360). Other suitable therapeutic agents for use in combination, either alternating or simultaneously, with the Dll4 antagonists of the invention may include agents that can block the biological activity of other VEGF family members such as VEGF-C and VEGF-D.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, areal measurements, etc.) but some experimental errors and deviations should be accounted for.

### Example 1

### Effect of Genetic Deletion of a Single Dll4 Allele on Blood Vessel Sprouting in the Developing Retinal Vasculature

An investigation was undertaken to determine the effects of Dll4 partial genetic deficiency on blood vessel sprouting during normal developmental retinal angiogenesis.

Animals. Velocigene™ technology (Valenzuela et al. (2003) Nat. Biotechnol. 21:652-9; U.S. Pat. No. 6,586,251, which references are specifically incorporated by reference in their entirety) was used to replace the entire Dll4 coding region with the β-galactosidase reporter gene in C57BL/6:129 hybrid mouse embryonic stem cells. Chimeric males were bred to ICR females. Dll4^{+/lacZ} mice backcrossed for 3 generations to ICR (87.5% ICR) were used for this study.

Histochemistry and Immunostaining. Mouse pups were humanely euthanized at P7. Eyes were enucleated, and retinas were dissected, fixed overnight with 4% paraformaldehyde, stained with FITC-labeled Griffonia simplicifolia (GS) lectin I (Vector Laboratories, Burlingame, Calif.), and flat-mounted. Images were taken by using a Nikon (Melville, N.Y.) microscope.

Quantifications. Measurements were performed using the Scion Image software. Each experimental condition was assayed at least in triplicate. Student's t-test and two-way analysis of variance were used to assess statistical significance.

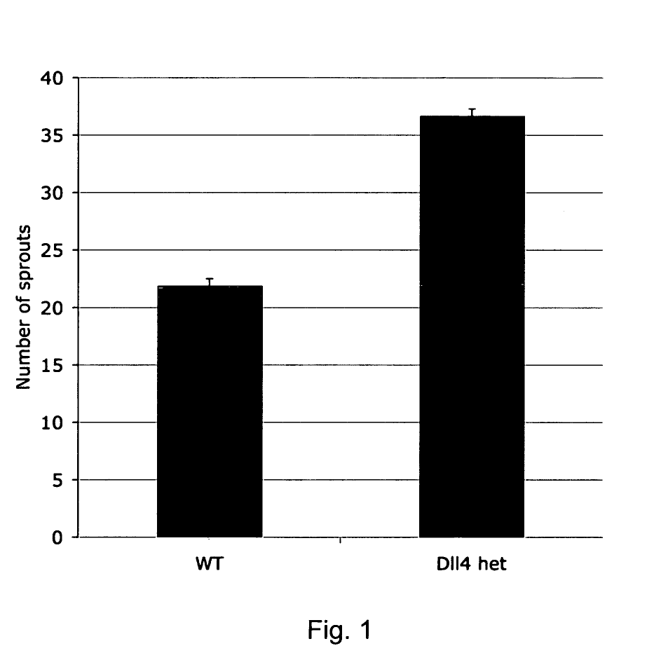

Results. Peripheral plexus was much denser in Dll4^{+/lacZ} mice than in wild-type littermates. Higher-power views highlighted that the denser peripheral plexus in the retinas of Dll4^{+/lacZ} mice consisted of capillaries that were larger in diameter, more highly interconnected and hyperfused, so that in some areas the vessels coalesced to form a syncytium, and, in addition, that there were many more sprouts at the growing front (FIG. 1); filopodia also were observed in more interior portions of the plexus at a higher than normal frequency. Quantitative analyses revealed that, compared to wild type controls, retinas of Dll4^{+/lacZ} mice showed a 68% increase in the number of sprouts at the growing front of the superficial retinal vasculature (FIG. 1), as well more than a two fold increase in the number of capillary interconnections per unit area, resulting in a significant increase in the vascular coverage. Despite the marked morphologic changes, intravascular injection of fluoresceinated lectin completely filled the developing superficial vascular plexus, except for the filopodia extending from the tip cells, in Dll4^{+/lacZ} mice as in wild-type mice, indicating that all components of the developing vasculature had lumens and were functional.

### Example 2

### Effect of Dll4/Notch Inhibition with Dll4-Fc or Anti-Dll4 Antibody on the Developing Retinal Vasculature

An investigation was undertaken to determine the effects of Dll4/Notch signaling pharmacological inhibition on endothelial cell proliferation and blood vessel sprouting during normal developmental retinal angiogenesis.

Antibodies and reagents. Dll4-Fc comprises the extracellular domain of mouse or human Dll4 and the Fc part of human IgG. Dll4-Fc was expressed in CHO cells and affinity purified by protein-A chromatography. Anti-Dll4 antibody was produced by immunization of rabbits with recombinant mDll4-hFc. The anti-serum was partially purified by protein-A chromatography prior to use.

Animals. C57/B16 mice (Taconic) were used to study the effect of Dll4-Fc or neutralizing Dll4 antibody on developing retinal vasculature.

Intravitreal microiniections. Intravitreal microinjections (30-100 nl) of the research compounds were made between the equator and the corneal limbus by using a Drummond Scientific (BroPA) nanoinjector equipped with a glass needle.

BrdU labeling. Proliferating cells were labeled by administration of BrdU (1 mg/kg i.p.) 20 h after intravitreal injection of hFc or Dll4-Fc. Retinas were harvested 4 h later and stained with ant-BrdU (Dako North America, Inc., Carpinteria, Calif.) and VE-Cadherin (BD PharMingen, San Diego, Calif.) antibodies.

Results. To study the effect of local intraretinal deficiency in Dll4/Notch signaling, and not secondary to an undetected systemic abnormality, a soluble version of Dll4 (termed Dll4-Fc) that binds Notch receptors without activating them, thereby blocking the actions of endogenous Dll4, or a neutralizing polyclonal antibody specific for the extracellular domain of Dll4, was injected into the vitreous of wild-type mice. Three days after intraocular administration of either Dll4 blocker, the retinal vessels exhibited morphologic changes that closely resembled the vascular abnormalities found in Dll4^{+/lacZ} mice, including dramatically increased blood vessel density and vessel caliber. Moreover, these characteristic morphologic changes occurred rapidly, being clearly evident within 24 hours.

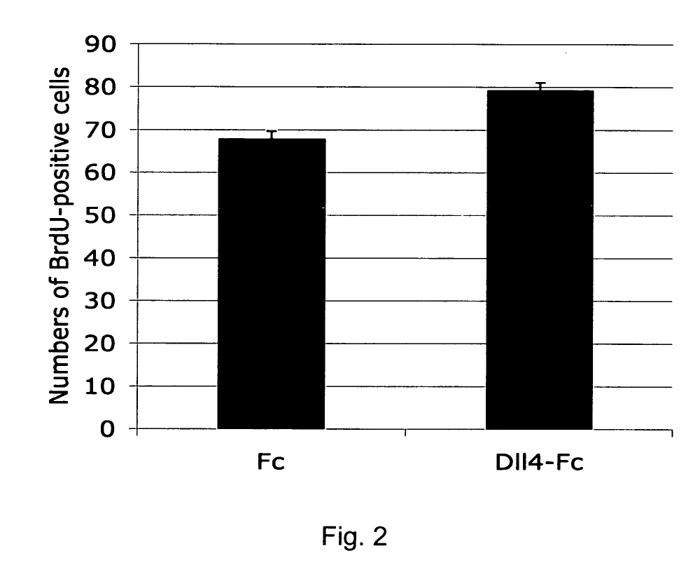

BrdU labeling also showed an increase in endothelial cell proliferation within 24 h of Dll4 blockade (FIG. 2). The observed ˜17% increase in the proliferation rate could yield more than 50% increase in cell number within three to four doubling times.

### Example 3

### Effect of Dll4-Fc and Anti-Dll4 Antibody on Retinal Vascularization, Perfusion and Vascular Abnormalities in Mice with OIR

An investigation was undertaken to determine the effects of the Dll4-Fc and anti-Dll4 antibody on the growth of retinal vessel, formation of vascular abnormalities and retinal perfusion in oxygen-induced ischemic retinopathy (OIR).

To determine whether Dll4/Notch signaling plays a role in pathologic angiogenesis as well as during normal development, the OIR model was utilized. In the OIR model, exposure of mouse pups to hyperoxia at P7 results in a rapid obliteration of capillaries in the central retina. Following return to room air at P12, the avascular zone becomes severely hypoxic, which in turn elicits extensive abnormal neovascularization, characterized by the ectopic growth of vessels into the vitreous (epiretinal vascular ‘tufts’) and the formation of abnormal arteriovenous shunts; central parts of the retina remain largely avascular for an extended period.

Animals and OIR model. C57/B16 mice (Taconic) were used to study the effect of Dll4-Fc or neutralizing Dll4 antibody on retinal neovascularization in OIR. OIR was produced following the method developed by Smith et al. (Invest. Ophthalmol. Vis. Sci. 1994, 35:101-111). Briefly, mouse pups and their dams were placed into a 75% oxygen environment from postnatal days (P) 7 to P12, and then returned to room air. Exposure to hyperoxia induces rapid vasoobliteration in the central retina. When mice are returned to room air (P12), the loss of vessels from the central retina results in severe hypoxia/ischemia which in turn stimulates the pathological vascular changes described above.

To label patent blood vessels, 50 ml of Texas red-labeled Lycopersicon esculentum (LE) lectin (1 mg/ml; Vector Laboratories, CA) was injected into the left cardiac ventricle and allowed to circulate for 5 min.

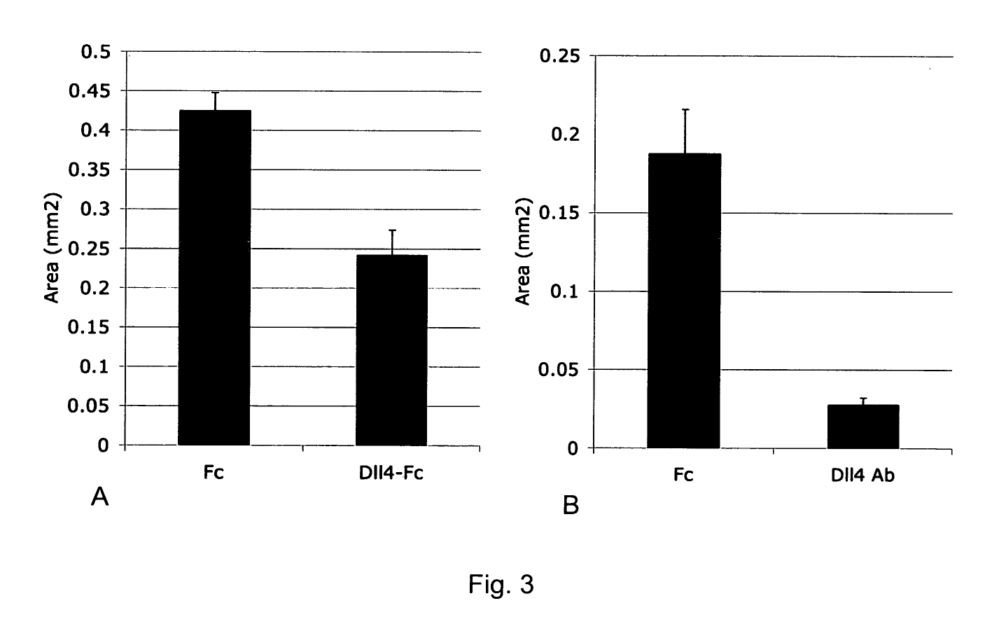

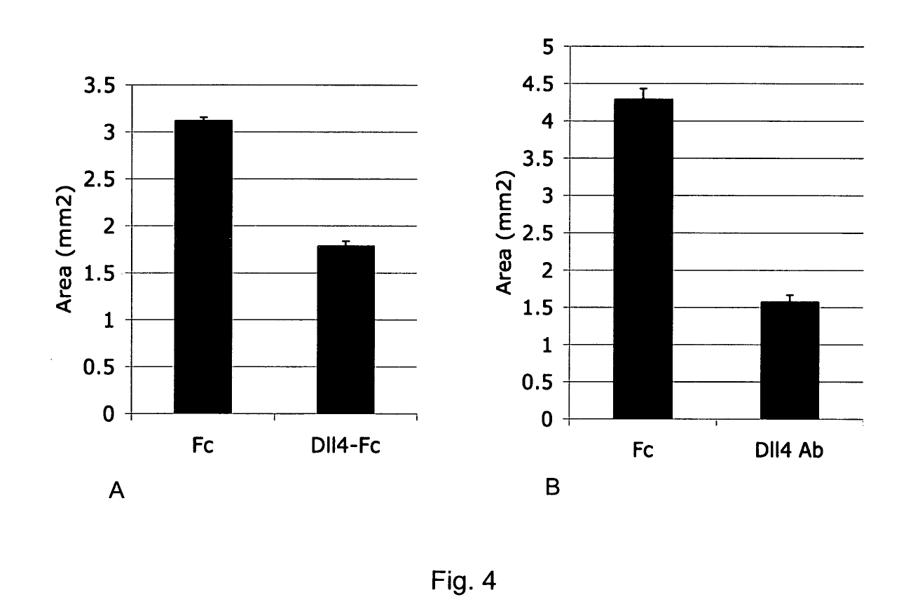

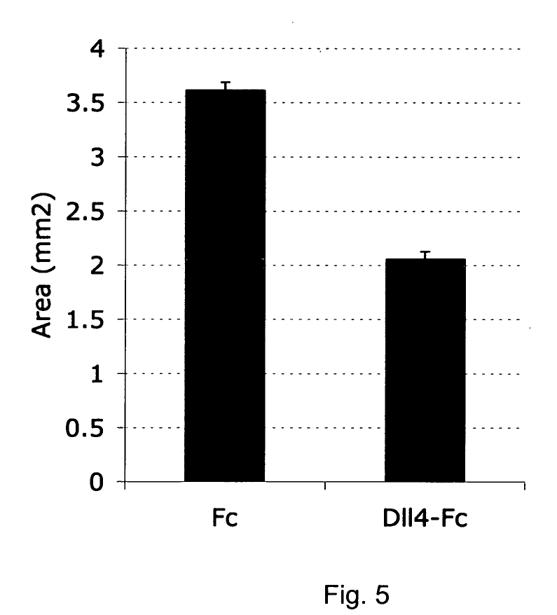

Results. Dll4/Notch inhibition with either Dll4-Fc or anti-Dll4 antibody ameliorated pathological neovascularization (FIG. 3A-B), stimulated growth of new blood vessel (FIG. 4A-B) and improved retinal re-perfusion (FIG. 5).

Dll4-Fc or anti-Dll4 antibody or a control protein (hFc) was injected intravitreally at P13, one day after the animals were returned to room air, well after vaso-obliteration was complete. When the retinas were evaluated at P17, administration of Dll4-Fc or anti-Dll4 antibody dramatically suppressed the ectopic growth of pathological neovascular tufts into the vitreous (FIG. 1), and also prevented the formation of abnormal arteriovenous shunts. The areas occupied by neovascular tufts was reduced by 43% in the retinas treated with Dll4-Fc and by 85% in the retinas treated with anti-Dll4 antibody both comparing to hFc treated control retinas.

Moreover, Dll4-Fc and anti-Dll4 antibody were found to stimulate more extensive sprouting of new vessels from capillaries and veins bordering the avascular zone, resulting in a more rapid re-growth of blood vessels into the central retina where the vasculature had been depleted thus reducing the avascular retinal area (FIG. 4A-B). The avascular areas were reduced by 43% in the retinas treated with Dll4-Fc and by 63% in the retinas treated with anti-Dll4 antibody. Particularly notable was the extensive capillary re-growth emanating from veins in the avascular zone of Dll4-Fc treated retinas. Thus, attenuation of Dll4/Notch signaling favored the extension of new vascular sprouts along the retinal surface, and obtunded the formation of epiretinal neovascularization, resulting in a more rapid reformation of the superficial vascular plexus. The new forming vessels were functional and exhibited improved retinal reperfusion as evident by 45% reduction of nonperfused areas in Dll4-Fc treated retinas (FIG. 5).

### Example 4

### Effect of Dll4-Fc and Anti-Dll4 Antibody on Retinal Hypoxia/Ischemia in the OIR Model

An investigation was undertaken to determine the effects of the Dll4-Fc and anti-Dll4 antibody on retinal hypoxia/ischemia in the OIR model of pathological neovascularization.

Excessive blood vessel growth under certain circumstances may interfere with normal blood circulation and reduce tissue oxygenation. To test whether Dll4/Notch inhibition can improve tissue oxygenation, HYPOXYPROBE™-1 (Chemicon) was used in a non-invasive assay to detect tissue hypoxia and determine the effect of Dll4-Fc treatment on retinal hypoxia/ischemia. HYPOXYPROBE™-1 was injected intraperitoneally at 100 mg/kg one hour prior to collecting the retinas for evaluation.

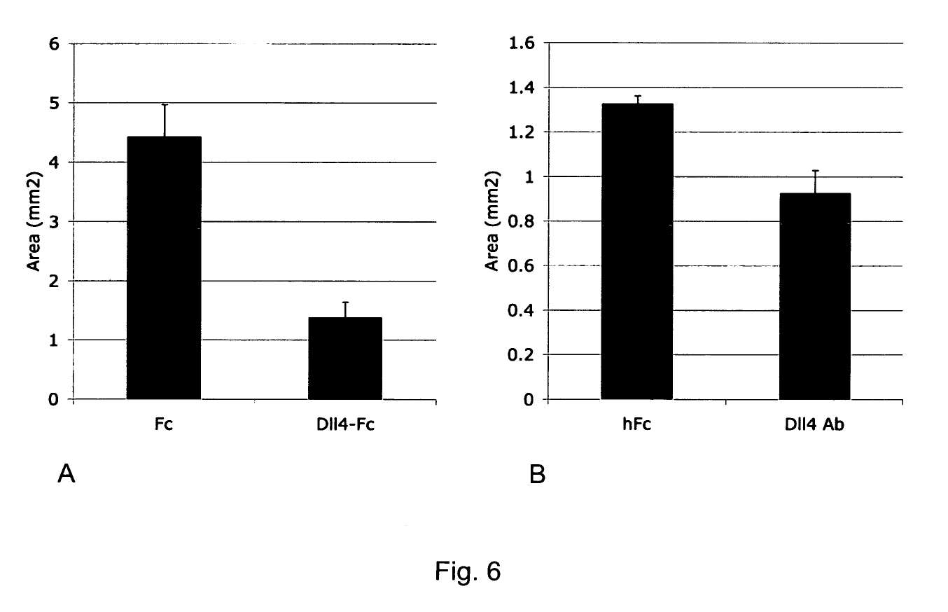

Results. The growth of functional new vessels following intravitreal administration of Dll4/Notch inhibitors effectively reduced tissue hypoxia/ischemia as evident by 69% and 30% reduction of hypoxyprobe-positive areas in Dll4-Fc or anti-Dll4 antibody treated retinas, respectively (FIG. 6A-B).

### Example 5

### Genetic Deletion of a Single Dll4 Allele Reduces Hyperoxia-Induced Vasoobliteration

An investigation was undertaken to determine the effects of Dll4 partial genetic deficiency on hyperoxia-induced blood vessel regression.

Animals. Dll4^{+/lacZ} mice backcrossed for 3 generations to ICR (87.5% ICR) were generated as described above. Due to a recessive (rd/rd) mutation the retinal photoreceptor cell layer starts to degenerate at P12 in ICR mice. Therefore, to obviate potential secondary effects of photoreceptor loss on the retinal vasculature, for evaluating later stages of retinal development and for all OIR experiments, Dll4^{+/lacZ} (87.5% ICR) mice were backcrossed to C57BL/6 to produce Rd/rd mice, which do not exhibit photoreceptor degeneration.

Mouse pups were placed into a 75% oxygen environment from postnatal days (P) 7 to P12. Retinas were harvested and retinal vasculature was analyzed in flat-mounts.

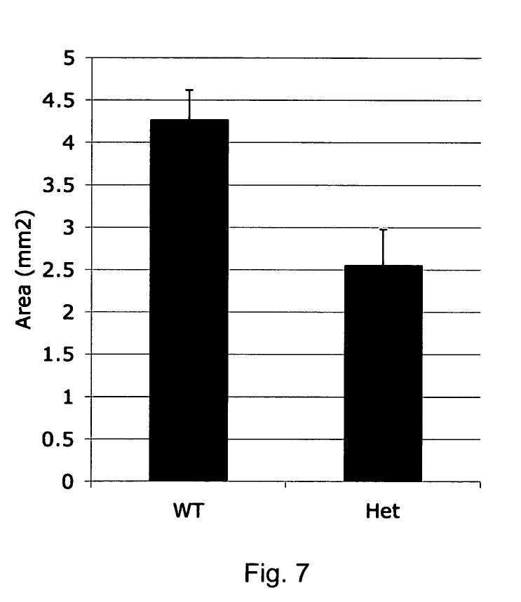

Results. Reduced expression of Dll4 in the retinal vasculature of Dll4^{+/lacZ} mice partially prevents hyperoxia-induced retinal blood vessel loss (FIG. 7). In the OIR model, exposure of mouse pups to hyperoxia at P7 results in a rapid occlusion and obliteration of capillaries in the central retina. To determine whether Dll4/Notch inhibition may have a protective effect on blood vessels, the effect of Dll4 partial genetic deficiency in Dll4^{+/lacZ} mice on hyperoxia-induced vasoobliteration was analyzed. Evaluating animals at P12 (so as to assess the extent of hyperoxic vaso-obliteration) it was found that vaso-obliteration was reduced by 40% in Dll4^{+/lacZ} mice comparing to wild-type littermate control animals, suggesting that Dll4 inhibitors may protect existing blood vessels from regression.

### Example 6

### Effects of Dll4-Fc and Anti-Dll4 Antibody on Hyperoxia-Induced Vaso-Obliteration

An investigation was undertaken to determine the effects of Dll4/Notch inhibition with Dll4-Fc and anti-Dll4 antibody on hyperoxia-induced blood vessel regression.

Animals. C57/B16 mice (Taconic) were used to study the effect of Dll4-Fc or neutralizing Dll4 antibody on oxygen-induced retinal vaso-obliteration. Intravitreal microinjections of the research compounds were performed at postnatal day 8. At postnatal day 9 pups were placed into a 75% oxygen environment. Retinas were harvested 24 hours later and retinal vasculature was analyzed in flat-mounts.

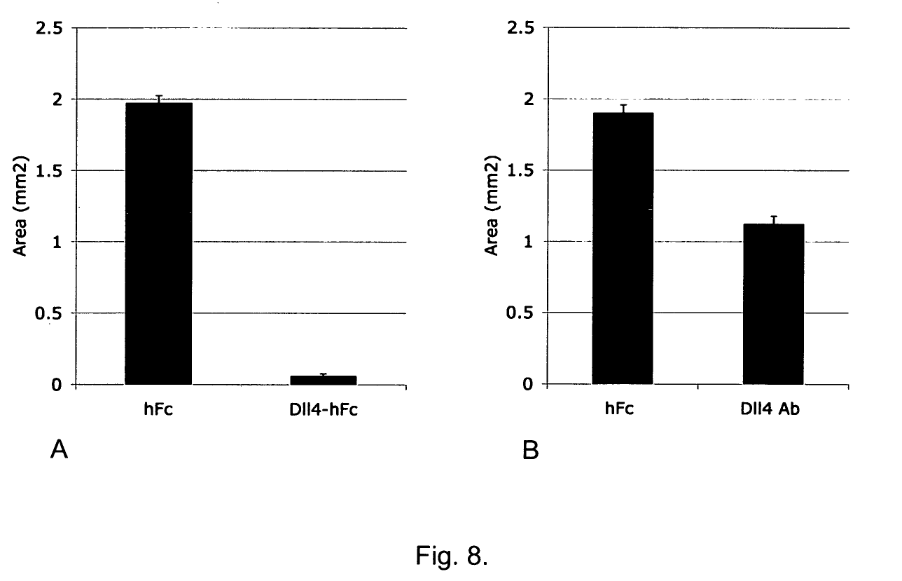

Results. Intravitreal injection of Dll4-Fc or anti-Dll4 antibody dramatically reduced areas of obliterating vasculature by 97% and 41% respectively (FIG. 8A-B).

### Example 7

### Effects of Dll4-Fc and Anti-Dll4 Antibody on Hyperoxia-Induced Blood Vessel Occlusion

An investigation was undertaken to determine the effects of Dll4/Notch inhibition with Dll4-Fc and anti-Dll4 antibody on hyperoxia-induced blood vessel occlusion. All procedures were performed as described above.

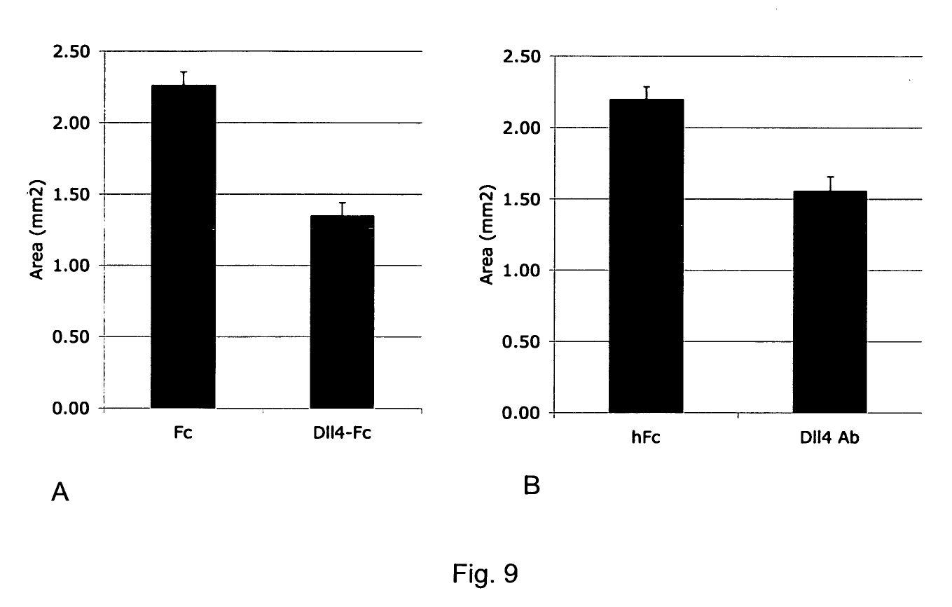

Dll4-Fc and anti-Dll4 antibody treatment reduced non-perfused retinal areas by 40% and 29% respectively (FIG. 9A-B).

### Example 8

### Effects of Systemic Administration of Dll4-Fc on Hyperoxia-Induced Blood Vessel Regression

An investigation was undertaken to determine the effects of systemic treatment with Dll4-Fc on hyperoxia-induced blood vessel regression.

4.1 mcg of hDll4-hFc or 5 mcg of human hFc control protein was injected intravitreally (ITV) or hDll4-hFc was injected intraperitoneally at a dose of 25 mg per kg of the body weight at P7. Pups were placed into a 75% oxygen environment at P8 and retinal vasculature was analyzed at P9. All other procedures were performed as described above.

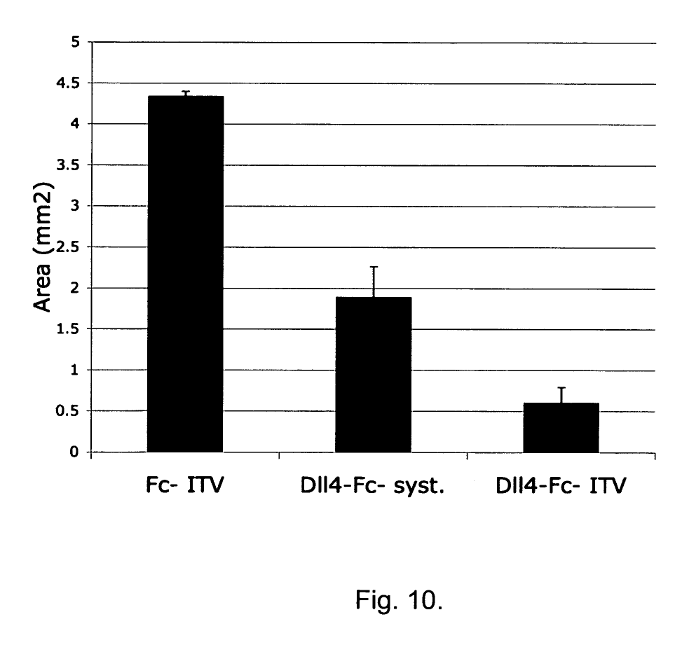

Results. Both local (intravitreal) and systemic (intraperitoneal) administration of Dll4-Fc reduced areas of obliterating vasculature by 86% and 56% respectively, indicating that independently of the route of administration Dll4 inhibitors can be effectively used to protect blood vessels from regression (FIG. 10).

What is claimed is:

1. A therapeutic method for treating an ischemic or vascular disorder, comprising administering an agent capable of inhibiting Dll4 activity to a subject in need thereof.

2. The therapeutic method of claim 1, wherein the ischemic or vascular disorder is an eye disease or disorder.

3. The therapeutic method of claim 1, wherein the Dll4 antagonist is an antibody or antibody fragment capable of blocking the binding of Dll4 to a Notch receptor.

4. The therapeutic method of claim 3, wherein the Dll4 antibody or antibody fragment is polyclonal or monoclonal.

5. The therapeutic method of claim 4, wherein the antibody or antibody fragment is humanized, chimeric, or is a fully human antibody or antibody fragment.

6. The therapeutic method of claim 5, wherein the antibody fragment is a single chain antibody, an Fab, or an F(ab′)₂.

7. The therapeutic method of claim 1, wherein the Dll4 antagonist comprises a fragment of Dll4, optionally connected to a multimerizing component.

8. The therapeutic method of claim 2, wherein the eye disease or disorder is characterized by the presence of abnormal blood vessels and/or loss of normal blood vessels or vessel function.

9. The therapeutic method of claim 8, wherein the eye disease is retinopathy of prematurity, ischemic retinopathy, retinal vein or artery occlusion, diabetic retinopathy, choroidal neovascularization, age related macular degeneration, corneal neovascularization, neovascular glaucoma or corneal transplantation.

10. The method of claim 1, wherein the subject is a human being.

11. The therapeutic method of claim 1, wherein the ischemic or vascular disorder is one of ischemic injury, cerebral ischemia, cardiac ischemia, ischemic conditions affecting the limbs and other organs or tissues, arteriovenous malformations, wound healing, organ or tissue transplantation, placental insufficiency, arterial narrowing and occlusion, atherosclerosis, and systemic or pulmonary hypertension.
